(19) United States
(12) Patent Application Publication
(10) Pub. No.: US 2006/0110825 A1
(43) Pub. Date: May 25, 2006

(54) Process for the preparation of stem cells from human muscle tissue and adipose tissue, and stem cell obtainable by this process

(76) Inventors: Giulio Alessandri, MILANO (IT); Arnaldo Caruso, Brescia (IT); Jose Sebastian Franzone, Torino (IT)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/510,622

(22) PCT Filed: Apr. 9, 2003
(86) PCT No.: PCT/EP03/03667

§ 371 Date: Jun. 27, 2005

(30) Foreign Application Priority Data
Apr. 10, 2002 (IT) TO2002A000311

(51) Int. Cl. C12N 5/08; A61K 35/30

(52) U.S. Cl. 435/368; 424/93.7

(57) ABSTRACT

The invention relates to a process for the preparation of human stem cells from muscle tissue or adipose tissue. The process provides for the incubation of cells obtained from a sample of muscle tissue or adipose tissue in a medium comprising BSA, bFGF, EGF, VEGF, LIF, heparin and usual inorganic salts, natural amino acids and vitamins necessary for the growth of mammalian cells. The invention relates also to the human muscle stem cells (hMSC) and human adipose tissue stem cells (hFSC) obtainable by this process.

This application claims priority under 35 U.S.C. 365 to International Application No. PCT/EP03/03667 (Apr. 9, 2003) and under 35 U.S.C. 119 to Italian application no. TO2002A000311 (Apr. 10, 2002), the disclosures of which are herein incorporated by reference.

The present invention relates to a process for the preparation of human stem cells from a sample of human adipose or muscle tissue, as well as human stem cells obtainable by this process.

In particular, the invention relates to the preparation of human stem cells of the muscle (hMSC) and of the adipose tissue (hFSC) from a sample of skeletal muscle tissue and of adipose tissue, respectively.

It is known that skeletal muscle has a regenerative capacity owing to the presence of immature progenitor muscle cells. All muscle fibres in fact contain cells capable of growing and differentiating to form muscle fibres; these cells are called satellite cells. Satellite cells are generally dormant and remain in a non-differentiated form under the basal lamina of the muscle fibre. A lesion of the muscle activates these cells, bringing them from the dormant phase into the growth phase. Some of these cells differentiate to form myocytes which, by fusing with one another, lead to the regeneration of a new muscle fibre, thus restoring normal muscle function. Another portion of the satellite cells remain in a non-differentiated form, returning the number of satellite cells in the muscle fibre to the original amount.

The present inventors have now developed a process which permits the production, starting from a sample of human muscle tissue, of stem cells which are even more non-differentiated than satellite cells, because they are capable of differentiating both to form satellite cells and also to form various cell elements, such as nerve cells (neurones, gliocytes, astrocytes), vascular cells (endothelium) and bone cells (osteoblasts).

The present inventors have also used the same preparation process on samples of adipose tissue and this has enabled them to obtain adipose tissue stem cells that are likewise capable of differentiating both to form muscle cells (smooth and striated) and to form nerve cells (neurones, gliocytes, astrocytes), vascular cells (endothelium) and bone cells (osteoblasts).

The present invention therefore relates firstly to a process for the preparation of human stem cells from a sample of human adipose or muscle tissue, comprising the steps of:

a) preparing a cell suspension from a sample of human adipose or muscle tissue;

b) recovering the cells from the cell suspension; and

c) incubating these cells in a medium comprising BSA, bFGF, EGF, VEGF, LIF, heparin and usual inorganic salts, natural amino acids and vitamins necessary for the growth of mammalian cells.

The medium used for the incubation of the cells is preferably the medium DMEM/F12 supplemented with: from 0.4% to 0.8% of BSA, from 5 to 20 ng/ml of bFGF, from 10 to 40 ng/ml of EGF, from 2.5 to 10 ng/ml of VEGF, from 5 to 20 ng/ml of LIF, from 1 to 20 μg/ml of heparin, from 1.8 to 3 mg/ml of glucose, from 2 to 2.5 mg/ml of NaHCO₃, from 2.5×10⁻³ to 7.5×10⁻³ M of Hepes, from 50 to 200 μg/ml of apotransferrin, from 10 to 30 μg/ml of insulin, from 3×10⁻⁴ to 7×10⁻⁴ M of putrescine, from 4×10⁻⁸ to 8×10⁻⁸ M of selenium, from 1×10⁻⁸ to 3×10⁻⁸ M of progesterone.

The easy accessibility of samples of muscle and of adipose tissue makes these tissues an ideal source for the isolation and growth of stem cells.

The present invention relates also to a human muscle stem cell (hMSC) obtainable by means of the process described above in which a sample of human skeletal muscle tissue is used as the starting tissue.

Yet another subject of the present invention is a human adipose tissue stem cell (hFSC) obtainable by means of the process described above in which a sample of human adipose tissue is used as the starting tissue.

In order to obtain hMSC cells, step a) of the process according to the invention preferably comprises the digestion of the sample of human skeletal muscle tissue with trypsin.

In addition, according to a preferred embodiment for obtaining hMSC cells, the incubation step c) comprises:

c₁) resuspending the cells recovered from the cell suspension of step a) in the growth medium defined above;

c₂) incubating the cell suspension obtained in the previous step inside a container for cell cultures, which has previously been treated with type I collagen, for from 18 to 24 hours at a temperature of approximately 37° C. and in a 5% CO₂ atmosphere;

c₃) removing the growth medium from the container and replacing it with an identical freshly prepared growth medium; and

c₄) incubating for a further 48 to 72 hours, thereby obtaining the formation of small roundish cells adhering to the walls of the container, the small adherent roundish cells being human muscle stem cells (hMSC).

Alternatively, according to a preferred embodiment for obtaining hFSC cells, the incubation step c) comprises:

c₁) resuspending the cells recovered from the cell suspension of step a) in the growth medium defined above;

c₂) incubating the cell suspension obtained in the previous step inside a container for cell cultures, which has previously been treated with type I collagen, for from 18 to 24 hours at a temperature of approximately 37° C. and in a 5% CO₂ atmosphere;

c₃) recovering the cells not adhering to the walls of the container and resuspending them in freshly prepared growth medium as defined above;

c₄) placing the cell suspension obtained in the previous step in a second container for cell cultures which has not previously been treated with collagen and cultivating the cells therein for from 7 to 10 days, thereby obtaining the formation of floating cell aggregates, the cells of these aggregates being human adipose tissue stem cells (hFSC).

In this embodiment, steps c₂) and c₃) are preferably repeated twice more—for a total of 3 changes of medium—before proceeding to the subsequent step c₄) of incubation in the container not treated with collagen.

Owing to their capacity to differentiate to form a multiplicity of various cell types, the stem cells hMSC and hFSC of the present invention can be used in a variety of therapeutic applications, such as:

the treatment of ischaemic tissue after thrombotic or traumatic phenomena or the repair of vascular damage caused by traumatic phenomena or of atherosclerotic origin (for these applications the stem cells can be engineered by the introduction into their genome of angiogenic factors, such as, for example, VEGF (“vascular endothelial growth factor”));

the regeneration of striated muscle tissue;

the regeneration of skeletal muscle tissue owing to traumatic events;

the cell treatment of myocardial infarct;

the regeneration of bone tissue and cartilaginous tissue;

in co-transplantation with other stem cells, such as, for example, medullary or neuronal cells, for supporting establishment and growth and promoting the regeneration of mesenchymal tissue (bone, cartilage, smooth and vascular muscle);

in the establishment of grafted bone tissue and, in general, in all conditions that require the establishment and growth of cells and tissue in the human organism;

the production of growth and/or trophic factors for cells of various origins and sources;

the production of hormones for therapeutic purposes in humans;

tissue bioengineering;

the regeneration of peripheral nerves;

the treatment of multiple sclerosis;

the regeneration of central nervous tissue;

the treatment of Parkinson's disease and Alzheimer's disease.

Isolation of hMSC

A bioptic sample of human skeletal muscle, after being weighed and preferably catalogued and recorded, is transferred into a culture petri dish and broken up finely with a bistoury into fragments of approximately 1 mm³ or less.

After the addition of PBS (isotonic phosphate buffer) and antibiotics, the fragments are transferred into a conical test tube and washed 3 times with PBS by light centrifuging at 200 rpm at 4° C. When the final washing operation has been completed and the supernatant has been discarded, a solution of 0.25% (weight/volume) trypsin and 0.25% EDTA (ethylenediaminetetraacetic acid) is added to the fragments. The volume quantity of trypsin to be added is calculated relative to the volume of fragmented tissue: for approximately 0.5 ml of tissue, approximately 3 ml of enzyme solution are added. The test tube is then transferred to a bath maintained at a constant temperature of 37° C. and is incubated for approximately 2 hours with slight agitation.

When the incubation of the fragments with trypsin is complete, the test tube is left to stand for approximately 10 minutes at ambient temperature in order to cause all of the undigested material to settle at the bottom of the test tube. The cells in suspension are sucked up with a Pasteur pipette and transferred into a fresh test tube containing the same volume of DMEM medium to which 10% FCS (foetal calf serum) has been added, this being used to block the action of the trypsin. The cells are then recovered by centrifuging at 1000 rpm for 10 minutes. The pellet obtained is subsequently washed 3 times with PBS by centrifuging in order to remove all of the FCS. Finally, the cell pellet obtained is resuspended in a growth medium for stem cells called HUMAN-G which is composed of DMEM/F12 medium (Gibco) containing: 0.8% of BSA (Bovine Serum Albumin), 10 ng/ml of bFGF (basic Fibroblast Growth Factor), 20 ng/ml of EGF (Epidermal Growth Factor), 5 ng/ml of VEGF (Vascular Endothelial Growth Factor), 10 ng/ml of LIF (Lymphocyte Inhibitor Factor), 10 μg/ml of heparin, 2.4 mg/ml of glucose, 2.25 mg/ml of NaHCO₃, 5×10⁻³ M of Hepes, 100 μg/ml of apotransferrin, 25 μg/ml of insulin, 6×10⁻⁴ M of putrescine, 6×10⁻⁸ M of selenium, and 2×10⁻⁸ M of progesterone.

The cells are then sown in a T 25 culture flask previously treated with type I collagen in order to promote cell adhesion. The flask is then incubated for 18-24 hours in an incubator at 37° C. with 5% of CO₂. At the end of the incubation period, the medium is removed and replaced with an identical freshly prepared HUMAN-G medium; the flask is then returned to the incubator for a further 48-72 hours.

The adhering cells in the culture flask are initially composed of a population of small spindle-shaped cells (that is to say, the satellite cells of the striated muscle), while the cells in suspension in the medium are generally red corpuscles or dead cells and are readily removed by suction. After approximately 48-72 hours' incubation, small roundish cells, that is to say, the muscle stem cells (hMSC), start to appear at the bottom of the culture together with the satellite cells.

These small roundish cells occur only when using the culture medium described above. In fact, with other growth media known in the art these small roundish cells do not develop.

After approximately 1-2 weeks from the initial sowing, the presence of cells in suspension, in addition to the spindle-shaped satellite cells and the small roundish cells, starts to be observed. These cells are not capable of multiplying autonomously but their number nevertheless increases as the culture progresses, suggesting that they originate from the small roundish cells. Therefore the cells in suspension do not represent another cell population but probably an intermediate stage of differentiation of the adhering stem cells. After approximately one month's culture, it is possible to obtain muscle stem cells (hMSC) in a reasonable amount. Under these experimental conditions, the hMSC can multiply in culture and can reach reasonable numbers (2-3×10⁶) without showing any particular signs of morphological variation at least after 3 months' culture. After this period, growth decreases and cells having the very elongate morphology characteristic of differentiated muscle cells start to appear. In addition, if the cells in suspension are removed from the hMSC culture and are cultivated on proteins of the basal membrane, such as laminin, they are able to differentiate to form cells of nervous origin (astrocytes, neurones) that is to say, differing from the original muscle tissue.

Isolation of hFSC

A sample of adipose tissue is weighed, transferred to a culture plate and washed with large amounts of PBS. After this operation, the adipose tissue, being generally very loose, does not require the mechanical breaking-down with bistouries which is necessary for muscle tissue. It is therefore readily broken down by the mechanical action of resuspension with a Pasteur pipette. The tissue is subsequently washed with PBS and transferred into a test tube and left to stand at ambient temperature for approximately 10 minutes. This procedure enables all of the floating fatty tissue to rise to the surface, while the connective component settles at the bottom of the test tube. The fatty component is then recovered with a pipette and transferred to a fresh test tube containing a 0.25% collagenase solution. The amount of enzyme solution to be added to the adipose tissue depends on the amount of material to be processed: for approximately 1 ml of fat, approximately 2 ml of enzyme solution are added. The material is incubated for approximately 2 hours at 37° C. with slight agitation, after which the digested tissue is washed 2 or 3 times with PBS by centrifuging at 1000 rpm for 10 minutes. The cell pellet obtained is resuspended in PBS and the cell suspension obtained is filtered (filter having a porosity of 30 μm) in order to remove all of the vascular fragments which are present in large amounts in the adipose tissue and which often have dimensions larger than 30 μm. All of the cells or the cell microaggregates having dimensions smaller than 30 μm are recovered by centrifuging (1000 rpm for 10 minutes), the supernatant is discarded and the cell pellet is resuspended in HUMAN-G medium as described above with regard to the production of hMSC, with the only variant that the concentrations of LIF and VEGF are 20 ng/ml and 10 ng/ml, respectively. The cells are then sown in a T 25 flask treated with collagen and incubated at 37° C. in a 5% CO₂ atmosphere for approximately 18-24 hours. The differentiated cells, the vascular fragments and the fibroblasts present in the preparation adhere to the culture flask, while all of the dead cells and the non-differentiated cells (that is to say, stem cells) continue to float in the medium. This procedure is generally repeated 3 times in order to be reasonably sure that all of the differentiated cells have been removed from the preparation. At the end of the final removal operation, the non-adhering cells are centrifuged at 500 rpm, resuspended in fresh HUMAN-G and sown in T 25 flasks not treated with collagen. After approximately 7-10 days from initial culturing, formations of several aggregate cells which float in the culture medium are observed. These cells are hFSC. After approximately 30-45 days' culture, the amount of stem cells reaches a reasonable number (approximately 2-3×10⁶)

Phenotypic and Functional Characterization of hMSC

An analysis was carried out in respect of the expression of various markers on the hMSC cells according to the invention.

By using techniques of cytofluorimetry, the positivity of the hMSC cells to CD34 and Bcl-2+, which are markers for which murine MSC were found to be positive in previous studies, was confirmed. Positivity to KDR/Flk-1 and Sca-1, that is to say, markers that are also found on bone marrow stem cells, was also confirmed.

The expression of muscle markers, such as desmine and myogenin, was evaluated by immunohistochemical and immunofluorescence studies.

In order to evaluate the characteristics of differentiation of hMSC to form cells of nervous origin, the neuronal phenotypic markers were analyzed at various culture times. The cells were cultivated for from 7 to 24 days on a substrate of laminin in the presence of culture medium without growth factors. After approximately 7-10 days, the presence of GAD, a marker for GABAergic neurones, was detected, which indicates differentiation to form neurones of the peripheral nervous system. It was also observed that hMSC was positive to GFAP, which suggests differentiation to form cells of the glia (gliocytes).

After approximately 21 days, an analysis was carried out in respect of the presence of neurofilaments-M (NFM), which was found to be positive only to a limited extent.

The differentiation of the hMSC cells to form smooth and striated muscle cells was analyzed using desmine antibodies. For that purpose, the cells were cultivated on collagen substrates in medium without growth factors in the presence of 3% FCS.

Differentiation to form bone cells (osteoblasts) was analyzed by assessing the presence of osteocalcin, a protein which is specifically produced by osteoblasts.

Because the literature has reported the existence of a progenitor common to the endothelial cells and the muscle cells of vessels, the observed capacity of hMSC to differentiate to form muscle tissue cells would suggest that these cells are also capable of differentiating to form endothelial phenotype.

Because the hFSC of the present invention are of the same mesenchymal origin as the hMSC, this also suggests that the same differentiating abilities described above contained in the hMSC are also present in the hFSC.

Medicaments use differentiable stem cells made according to the process described herein as the active ingredient in combination with one or more medically acceptable auxiliary components. The auxiliary components include pharmaceutically acceptable bases, stabilizers, antiseptics, preservatives, emulsifiers, suspending agents, solvents, solubilizers, lubricants, correctives, colorants, aromatics, soothing agents, vehicles, binders, thickeners (viscosity increasing agent), and buffers, and the like. Specirid examples thereof inlcude calcium carbonate, lactose, sucrose, sorbitol, mannitol, starch, amylopectin, cellulose derivatives, gelatin, cacao butter, distilled water for injection, sodium chloride solution, Ringer solution, glucose solution, human serum albumin (HSA), and the like. The amount of auxiliary components may be chosen within a pharmaceutically acceptable range depending on the form of the pharmaceutical composition and the like.

The dose of the pharmaceutical composition of the present invention may be determined depending on the state, age, sex, and body weight of the patients. Dosage generally will fall in the range used for other active therapeutic proteins, as is understood by the existing level of ordinary skill in the art using no more than routine experimentation.

The method of administration may be chosen depending on the state of the patients from various methods of administration such as oral, intramuscular, intraperitoneal, intradermal, subcutaneous, intravenous, intraarterial, or rectal administration. Preferably, the present therapeutic composition is administered by injection. Stem cells according to the invention are preferably used in medicaments at a concentration within the range of 1×10⁵ to about 5×10⁶ cells/ml in any physiologically and pharmaceutically acceptable salt.

1. A process for the preparation of human stem cells from a sample of human adipose or muscle tissue, said human stem cells being capable of differentiating into nerve cells, vascular cells and bone cells, the process comprising the steps of:
a) preparing a cell suspension from a sample of human adipose or muscle tissue;
b) recovering the cells from the cell suspension; and
c) incubating these cells in a medium comprising BSA, bFGF, EGF, VEGF, LIF, heparin and usual inorganic salts, natural amino acids and vitamins necessary for the growth of mammalian cells.

2. A process according to claim 2, wherein the medium is DMEM/F12 supplemented with: from 0.4% to 0.8% of BSA, from 5 to 20 ng/ml of bFGF, from 10 to 40 ng/ml of EGF, from 2.5 to 10 ng/ml of VEGF, from 5 to 20 ng/ml of LIF, from 1 to 20 μg/ml of heparin, from 1.8 to 3 mg/ml of glucose, from 2 to 2.5 mg/ml of NaHCO₃, from 2.5×10⁻³ to 7.5×10⁻³ M of Hepes, from 50 to 200 μg/ml of apotransferrin, from 10 to 30 μg/ml of insulin, from 3×10⁻⁴ to 7×10⁻⁴ M of putrescine, from 4×10⁻⁸ to 8×10⁻⁸ M of selenium, from 1×10⁻⁸ to 3×10⁻⁸ M of progesterone.

3. A process according to claim 1, wherein the tissue sample is a human skeletal muscle sample and the stem cells are human muscle stem cells (hMSC).

4. A process according to claim 3, wherein step a) comprises the digestion of the skeletal muscle sample with trypsin.

5. A process according to claim 3, wherein step c) comprises:
c₁) resuspending the cells recovered from the cell suspension of step a) in the growth medium as defined in claim 1;
c₂) incubating the cell suspension obtained in the previous step inside a container for cell cultures, which has previously been treated with type I collagen, for from 18 to 24 hours at a temperature of approximately 37° C. and in a 5% CO₂ atmosphere;
c₃) removing the growth medium from the container and replacing it with an identical freshly prepared growth medium; and
c₄) incubating for a further 48 to 72 hours, thereby obtaining the formation of small roundish cells adhering to the walls of the container, the small adherent roundish cells being human muscle stem cells (hMSC).

6. Human muscle stem cells (hMSC) capable of differentiating into nerve cells, vascular cells and bone cells, obtained by a process according to claim 3.

7. A process according to claim 1, wherein the tissue sample is a human adipose tissue sample and the stem cells are human adipose tissue stem cells (hFSC).

8. A process according to claim 7, wherein said step c) comprises:
c₁) resuspending the cells recovered from the cell suspension of step a) in the growth medium as defined in claim 1;
c₂) incubating the cell suspension obtained in the previous step inside a container for cell cultures, which has previously been treated with type I collagen, for from 18 to 24 hours at a temperature of approximately 37° C. and in a 5% CO₂ atmosphere;
c₃) recovering the cells not adhering to the walls of the container and resuspending them in freshly prepared growth medium as defined in claim 1;
c₄) placing the cell suspension obtained in the previous step in a second container for cell cultures which has not previously been treated with collagen and cultivating the cells therein for from 7 to 10 days, thus obtaining the formation of floating cell aggregates, the cells of these aggregates being human adipose tissue stem cells (hFSC).

9. A process according to claim 8, wherein steps c₂) and c₃) are repeated twice more.

10. Human adipose tissue stem cells (hFSC) capable of differentiating into nerve cells, vascular cells and bone cells, obtainable by a process according to claim 7.

11. Stem cells according to claim 6 for use in the regeneration of tissue selected from the group consisting of bone tissue, cartilaginous tissue, endothelial tissue, smooth muscle tissue, striated muscle tissue and nerve tissue.

12. Stem cells according to claim 6 for use in the treatment of ischaemic tissue, in the repair of vascular damage, in the cell treatment of myocardial infarct, in co-transplantation with other stem cells or tissues, in the production of growth and/or trophic factors, in the production of hormones, in tissue bioengineering, in the regeneration of peripheral nerves, in the treatment of multiple sclerosis, in the treatment of myocardial infarct, in the treatment of Alzheimer's disease or in the treatment of Parkinson's disease.

13. Use of stem cells according to claim 6 for the preparation of a medicament for the regeneration of tissue selected from the group consisting of bone tissue, cartilaginous tissue, endothelial tissue, smooth muscle tissue, striated muscle tissue and nerve tissue.

14. Use of stem cells according to claim 6 for the preparation of a medicament for the treatment of ischaemic tissue, the repair of vascular damage, the cell treatment of myocardial infarct, co-transplantation with other stem cells or tissues, the regeneration of peripheral nerves, the treatment of multiple sclerosis, the treatment of myocardial infarct, the treatment of Alzheimer's disease or the treatment of Parkinson's disease.
